(19) United States
(12) Patent Application Publication
(10) Pub. No.: US 2023/0399679 A1
(43) Pub. Date: Dec. 14, 2023

(54) SYSTEM AND METHOD FOR LIGAND-LIMITED NORMALIZING POLYMERASE CHAIN REACTION (LLN-PCR)

(71) Applicant: GTSeek, LLC, Twin Falls, ID (US)

(72) Inventor: Nathan Campbell, Twin Falls, ID (US)

(21) Appl. No.: 18/332,520

(22) Filed: Jun. 9, 2023

Related U.S. Application Data
(60) Provisional application No. 63/366,140, filed on Jun. 9, 2022.

(51) Int. Cl. C12Q 1/6806; C12Q 1/686; C12Q 1/6853

(52) U.S. Cl. CPC C12Q 1/6806; C12Q 1/686; C12Q 1/6853

(57) ABSTRACT

A method for obtaining a normalized quantity of DNA amplicons includes adding to a volume of a sample comprising a target DNA segment, a first concentration of a forward primer. The method includes adding, to the volume of the sample, a second concentration of a second primer, wherein the second primer comprises an unlabeled reverse primer. The method includes adding a third concentration of the third primer, wherein the third primer is a ligand modified reverse primer complementary to a second end of the target DNA segment and comprises a normalizing ligand. The method further includes performing a polymerase chain reaction (PCR) on the volume of the sample to obtain a normalized concentration of ligand-tagged amplicons, wherein the PCR comprises one or more melt cycles, annealing cycles, and extension cycles.

### CROSS-REFERENCE TO RELATED APPLICATION AND PRIORITY CLAIM

This application claims priority under 35 U.S.C. § 119(e) to U.S. Provisional Patent Application No. 63/366,140 filed on Jun. 9, 2022, which is hereby incorporated by reference in its entirety.

### TECHNICAL FIELD

This disclosure relates generally to genetic testing and research, and in particular, systems and methods for a ligand-limited polymerase chain reaction (PCR) normalization.

### BACKGROUND

Polymerase chain reaction (PCR) synthesis, wherein a section of deoxyribonucleic acid (DNA) of interest within a sample (for example, a small volume of saliva, nasal mucus, or pond water) containing genomic DNA is replicated at scale through multiple iterations of a process of denaturing the DNA to separate strands of DNA, annealing the single stranded DNA so that oligonucleotide primers can bind to complementary sequences on the section of DNA, and extending the samples to allow a DNA polymerase to synthesize new strands of DNA (also referred to as amplicons), is a fundamental technique of genetic analysis.

Advances in analytical hardware, including, without limitation, next-generation sequencing platforms have catalyzed significant improvements in the rate at which DNA analyses, such as base-pair sequencing can be performed. To achieve these significant improvements in throughput, many analytical platforms rely on parallel processing, and analyzing libraries of numerous (for example, up to thousands of samples at once) amplicon-containing samples. In such analyses, a plurality of amplicon-containing samples that are deposited in the sample wells are processed by a flow cell. In many cases, the accuracy of quantitation and other sequencing is significantly improved if the contents of the respective sample wells are normalized—meaning that equal numbers of sequencing library constructs are present in each sample. In this way, the effects of variations in quality across original samples (for example, DNA taken from decaying animal specimens versus DNA taken from freshly harvested specimens) can be mitigated.

Historically, the general approach for preparing sample sets for sequencing has been to treat amplification (for example, PCR synthesis and reproduction of DNA of interest) tagging (i.e., attaching a marking molecule to amplicons) and normalization (i.e., standardizing the quantities of amplicons in each of the sample volumes) as separate and discrete processes, wherein an amplicon-containing sample would be generated, tagged, and then normalized either manually (for example, by manual normalization, where samples would be measured and then diluted to achieve common concentrations of amplicons), or by using a commercial normalization plate, where the chemistry in the plate would normalize the population of tagged amplicons in each sample well.

The historical approach to generating a plurality of normalized samples of an amplicon is to generate amplicons and then perform separate tagging and normalization processes to generate a plurality of separate volumes of a sample for parallel sequencing analyses. Examples of historical techniques for normalization of separate sample volumes include spectroscopy, size-restricted spectroscopy, and quantitative binding, wherein a sample plate contains chemistry binding approximately the same quantity of amplicons to the plate, and liquid containing surplus amplicons can be rinsed away using a wash buffer.

However, as the number of sample volumes required for analyses increases, these historical approaches present, at a minimum, efficiency problems in the separate rounds of pipetting and elution are required when tagging and normalization are performed as separate processes. Further, the use of proprietary supplies, such as quantitative binding plates (for example, SEQUALPREP™ plates) can present further bottlenecks on a throughput of a respective laboratory, which, depending on the application, may be unacceptable (for example, such as in laboratories performing time-sensitive tests for the COVID-19 coronavirus). Further, separately normalizing each sample as described above implies that the time spent normalizing samples of tagged amplicons scales with the number of samples to be sequenced, creates an undesirable throughput and performance bottleneck.

That is, the practical and technical benefits of sequencers that can rapidly and simultaneously read numerous samples (for example, thousands of samples) are diluted if the time to perform sample preparation is not also reduced.

Accordingly, expanding the range of techniques for normalizing quantities of amplicons across sample volumes remains a source of technical challenges and opportunities for improvement in the art.

### SUMMARY

This disclosure provides a system and method for a ligand-limited normalizing polymerase chain reaction (LLN-PCR).

In a first embodiment, a method for obtaining a normalized quantity of DNA amplicons includes adding to a volume of a sample comprising a target DNA segment, a first concentration of a forward primer, wherein the forward primer is complementary to a first end of the target DNA segment. The method further includes adding, to the volume of the sample, a second concentration of a second primer, wherein the second primer includes an unlabeled reverse primer having a first annealing temperature, and a binding site specific to a complement of a third primer. The method includes adding, to the volume of the sample, a third concentration of the third primer, wherein the third primer is a ligand modified reverse primer complementary to a second end of the target DNA segment and includes a normalizing ligand. The third primer has a second annealing temperature that is greater than the first annealing temperature. The third concentration is less than the second concentration. The method further includes performing a polymerase chain reaction (PCR) on the volume of the sample to obtain a normalized concentration of ligand-tagged amplicons, wherein the PCR includes one or more melt cycles, annealing cycles, and extension cycles. The annealing cycles are performed at a third annealing temperature that is closer to the second annealing temperature than to the first annealing temperature.

In a second embodiment, a normalization plate includes a substantially planar member including a plurality of sample wells, in which each sample well of the plurality of sample wells includes a volume of a dried sucrose solution bound to the sample well. The dried sucrose solution includes a first concentration of a forward primer. The forward primer is complementary to a first end of a target DNA segment, and a second concentration of a second primer. The second primer is a ligand modified reverse primer complementary to a second end of the target DNA segment, wherein the second primer comprises a normalizing ligand. The second primer has a second annealing temperature that is greater than an annealing temperature of a third primer.

Other technical features may be readily apparent to one skilled in the art from the following figures, descriptions, and claims.

Before undertaking the DETAILED DESCRIPTION below, it may be advantageous to set forth definitions of certain words and phrases used throughout this patent document. The term “couple” and its derivatives refer to any direct or indirect communication between two or more elements, whether or not those elements are in physical contact with one another. The terms “transmit,” “receive,” and “communicate,” as well as derivatives thereof, encompass both direct and indirect communication. The terms “include” and “comprise,” as well as derivatives thereof, mean inclusion without limitation. The term “or” is inclusive, meaning and/or. The phrase “associated with,” as well as derivatives thereof, means to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

Definitions for other certain words and phrases are provided throughout this patent document. Those of ordinary skill in the art should understand that in many if not most instances, such definitions apply to prior as well as future uses of such defined words and phrases.

### BRIEF DESCRIPTION OF THE DRAWINGS

For a more complete understanding of this disclosure and its advantages, reference is now made to the following description, taken in conjunction with the accompanying drawings, in which:

FIG. 1 illustrates an example of a sequencer-ready construct obtained by apparatus and methods according to this disclosure;

FIGS. 2A-2E illustrate, at a single segment level, aspects of an example method for performing ligand limited normalizing PCR according to various embodiments of this disclosure;

FIG. 3 illustrates the relative rates of PCR amplification of ligand tagged amplicons relative to untagged amplicons according to various embodiments of this disclosure;

FIG. 4 illustrates aspects of normalization in sequence reads obtained by methods according to various embodiments of this disclosure;

FIG. 5 illustrates an example of a normalization plate according to various embodiments of this disclosure; and

FIG. 6 illustrates operations of an example method according to various embodiments of this disclosure.

### DETAILED DESCRIPTION

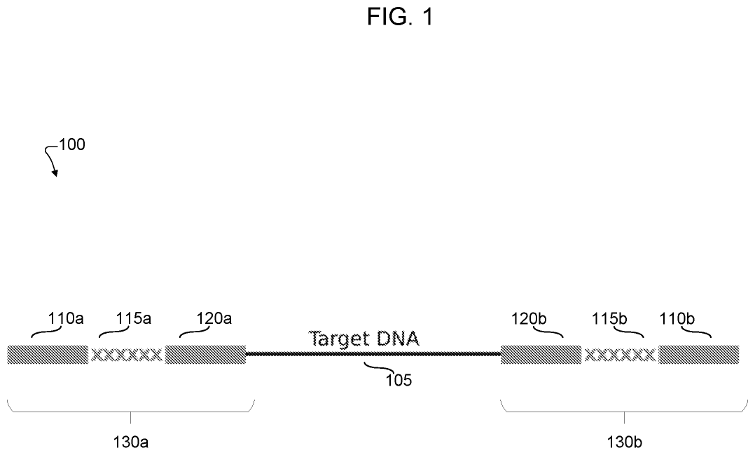

FIGS. 1 through 6, discussed below, and the various embodiments used to describe the principles of this disclosure in this patent document are by way of illustration only and should not be construed in any way to limit the scope of the disclosure. Those skilled in the art will understand that the principles of this disclosure may be implemented in any suitably arranged processing platform.

As noted in this disclosure, certain embodiments according to this disclosure include methods and an apparatus for obtaining normalized concentrations of labeled amplicons suitable from which the individual base pairs of a DNA sequence can be read in an apparatus (for example, an Ion Torrent sequencer or an Illumina sequencer) that requires the addition of platform-specific DNA adapter sequences. By iteratively adding fluorescently labeled terminators, identifying a base pair, and then removing the terminators, the sequence of a DNA strand can be read, for example, by using Illumina SBS technology. Alternatively, the Ion Torrent adds a single nucleotide base at a time to the flow-cell and detects the release of H+ ions as the DNA strand is copied. Although there are many possible strategies for NGS sequencing, any method for massively parallel sequencing will require DNA normalization prior to sequencing.

In next generation sequencers, multiple different strands of DNA from different sources, or comprising different parts of a larger DNA sequence, can be read simultaneously, and to differentiate the strands under analysis, indexing sequences may be added to the ends of the DNA under analysis to produce a labeled amplicon (also referred herein as a “construct”). In addition to target DNA for analysis, constructs contain additional unique, known sequences of DNA from which a plurality of read DNA sequences may be associated with its source.

Referring to the non-limiting example of FIG. 1, an example of a sequencer-ready construct 100 obtained by systems and methods according to this disclosure is depicted. The embodiment of the sequencer-ready construct 100 shown in FIG. 1 is for illustration only and other embodiments could be used without departing from the scope of the present disclosure.

According to certain embodiments, the construct 100 comprises a length of double-stranded deoxyribonucleic acid (DNA) having a plurality of sections. Embodiments of construct 100 may typically have a length in the range of 170-320 base pairs (Bp), though longer and shorter embodiments are possible, and are within the contemplated scope of this disclosure. The sections of construct 100 include a segment of target, or template DNA 105. According to various embodiments, the template DNA 105 includes a section of DNA (DNA obtained from a sample) of a plurality of segments of template DNA to be simultaneously read by a sequencer. In certain embodiments, the template DNA 105 is obtained by performing an initial PCR cycle on a sample containing the template DNA 105 to amplify the concentration of the template DNA 105 in the sample to an increased, but un-normalized concentration of the template DNA 105.

In some embodiments, the technical challenges associated with performing next generation sequencing include, without limitation, binding DNA for analysis to a flow cell, providing a mechanism to differentiate between analyzed DNA samples, and associating read sequences with originally-provided samples. As such, in some embodiments, the construct 100 further includes a first capture sequence 110a, which includes a first oligonucleotide configured to bind with a first complementary oligonucleotide on the surface of a flow cell. In one non-limiting example, a first capture sequence 110a is an Illumina P5 capture sequence. As shown in FIG. 1, the construct 100 further includes a second capture sequence 110b, which includes a second oligonucleotide configured to bind with a second complementary oligonucleotide on the surface of the flow cell, to form a ‘bridge’ of DNA that can be annealed and read by iteratively adding and removing fluorescent terminators. In one non-limiting example, a second capture sequence 110b is an Illumina P7 capture sequence.

To facilitate associating a read sequence of DNA with an original sample or part of a larger DNA sequence of which the sequence is a part, the construct 100 includes a first index sequence 115a and a second index sequence 115b. According to various embodiments, the first index sequence 115a comprises a section of DNA with an already known sequence, which binds at a first end, to first capture sequence 110a, and binds, at a second end to a first read primer site 120a. Collectively, first capture sequence 110a, first index sequence 115a, and first read primer site comprise a first adapter 130a. In this example, the second index sequence 115b comprises a second section of DNA with an already known sequence, which binds at a first end to second capture sequence 110b, and binds at a second end to a second read primer site 120b. Collectively, the second capture sequence 110b, second index sequence 115b, and second read primer site comprise a second adapter 130b.

According to some embodiments, the first read primer site 120a is a section of DNA, which, when annealed, forms a binding site for a first, forward primer for reading the DNA of construct 100. Similarly, second sequencing site 120b can be a second section of DNA, which, when annealed forms a binding site for a second, reverse primer for reading DNA of construct 100. Depending on embodiments, the construct 100 may be read either by paired-end or single-ended sequencing.

As discussed in greater detail herein, certain apparatus and methods according to the present disclosure provide a mechanism for obtaining normalized concentrations of construct 100, which allow sample normalization to be performed efficiently, accurately, and at scale by combining tagging and normalization into a single process.

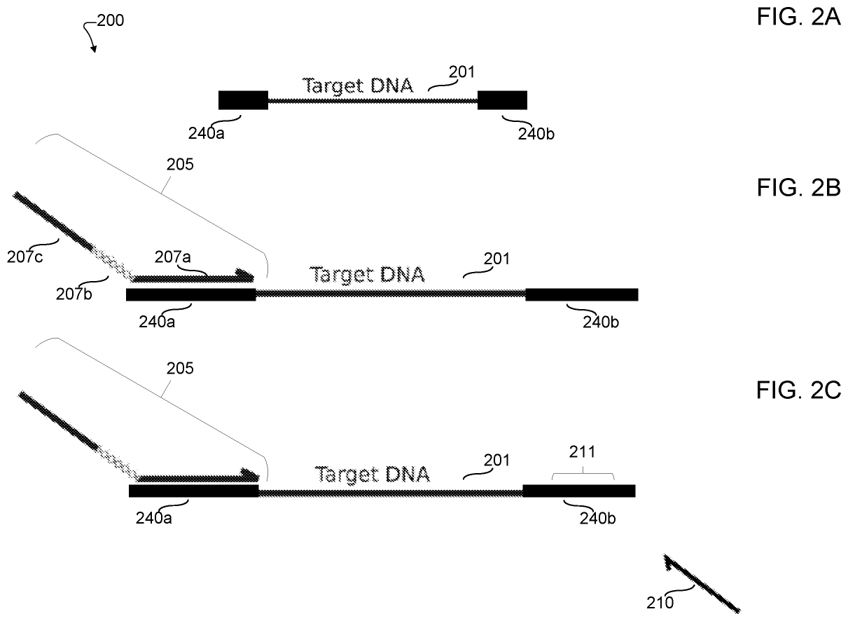

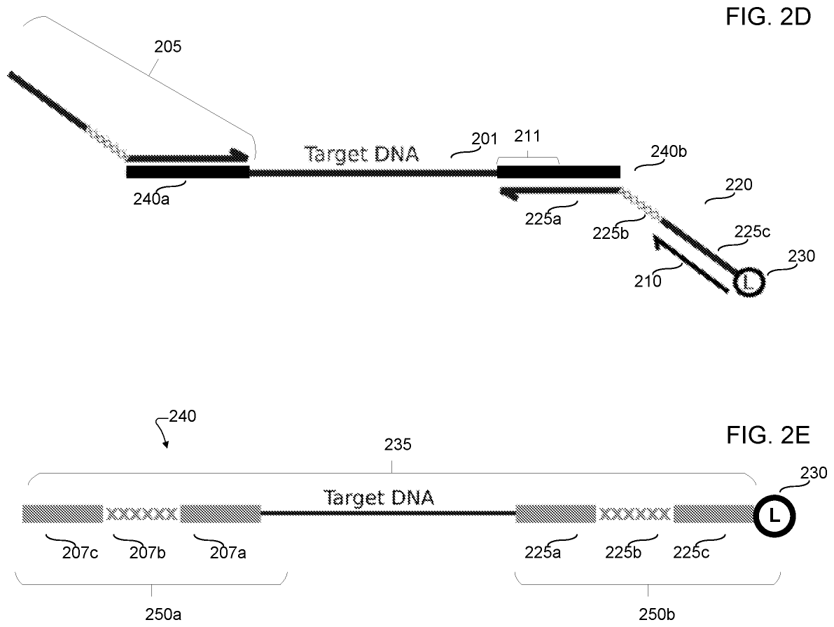

FIGS. 2A-2E illustrate, at a single segment level, aspects of an example method 200 for performing ligand limited normalizing PCR according to various embodiments of this disclosure. For convenience of cross reference, elements common to more than one of FIGS. 2A-2E are numbered similarly. The example method(s) shown in FIGS. 2A-2E are for illustration only and other steps and processes could be incorporated, removed, or depicted without departing from the scope of the present disclosure.

Referring to the example shown in FIG. 2A, in certain embodiments, a method 200 commences with an intermediate construct comprising a segment of target or template DNA 201 between a first adapter 240a (for example, an Illumina read1 primer site sequence such as site 120a in FIG. 1) and a second adapter 240b (for example, an Illumina paired-end primer sequence, such as site 120b in FIG. 1). In this example, the method 200 produces a normalized concentration of ligand labeled constructs of DNA sequences bounded by first adapter 250a and second adapter 250b. Typically, segment of target DNA 201 has a length between 75-150 base pairs, though embodiments according to this disclosure are not limited thereto. In some embodiments, a segment of the target DNA 201 is part of a larger sample of DNA at an unspecified concentration in a volume of liquid. In one example, the target DNA 201 may be a copy of a segment of DNA from a sample of interest (for example, DNA from some trout as part of a genotyping study of a body of water). In this example, the operations on the target DNA 201 described with reference to FIGS. 2A-2E are performed on a plurality of DNA segments to obtain a normalized concentration of labeled constructs (for example, construct 100 in FIG. 1).

Referring to the explanatory example of FIG. 2B, a first concentration of a first primer 205, is added to target DNA 201. According to various embodiments, a first primer 205 is a forward primer, which is provided at a concentration significantly exceeding that of a third, ligand-modified reverse primer. In some embodiments, the first primer 205 is provided at a concentration that is 10-50 times greater than that of the third primer. For example, where the ligand-modified reverse third primer is provided at a concentration of 8-12 nanomolar (nM), the first primer 205 can be provided at a concentration between 80 and 600 nM.

In certain embodiments, the first primer 205 is a user-designed primer in the range of 30-60 base pairs in length, though longer and shorter primers are possible and contemplated herein. As shown in the illustrative example of FIG. 2B, the first primer 205 can include a first binding site 207a, which comprises a section of DNA designed to bind to the start of target DNA 201 as a complement to the site of first adapter 240a. Further, the sequence of base pairs constituting first binding site 207a may comprise, some or all of a first read primer site (for example, first read primer site 120a in FIG. 1). According to various embodiments, first primer 205 further includes a first index sequence 207b (for example, first index sequence 115a) that operates as a label, or identifier, from which a read of the sequence of target DNA 201 can be associated with a specific sample.

Referring to the example of FIG. 2C, in certain embodiments, a concentration of a second primer 210 is added to the mixture containing an unspecified concentration of target DNA 201 and first primer 205. According to various embodiments, the second primer 210 is an unlabeled reverse primer targeting site (for example, second capture sequence 110b in FIG. 1). In certain embodiments, this unlabeled reverse primer targeting site is not added to the construct until the first two cycles of PCR have concluded and, therefore, this unlabeled reverse primer cannot bind to the construct until the third or subsequent PCR cycles. As with the first primer 205, the second primer 210 may be a user-designed primer. In the example shown in FIG. 2C, the primer 210 targets the P7 target capture sequence for Illumina sequencing instruments. In this example, the second primer 210 can have a length in the range of 15-30 base pairs, though embodiments of greater or shorter length are possible and within the contemplated scope of this disclosure. According to certain embodiments, the primer 210 binds to its complement at a lower temperature than the ligand-modified third primer. As with the first primer 205, the second primer 210 is added at a concentration designed to significantly exceed that of a third, ligand-modified primer. In various embodiments, the second primer 210 is provided at a concentration between 10-50 times that of the third primer. Thus, where the third, ligand-modified primer is provided at a concentration between 8-12 nM, the second primer 210 is provided at a concentration between 80-600 nM. The aforementioned ranges of concentrations are provided as non-limiting examples, and greater and lesser concentration ranges may be possible.

In certain embodiments, the second primer 210 is designed to bind to a third binding site that is complementary to site 225c and which will be added during the first two cycles of PCR (for example, a binding site comprising all or part of Illumina P7 capture site sequence 110b) of target DNA 201. In addition to comprising a section of DNA designed to bind with second binding site 225c, the second primer 210 is designed such that the second primer 210 has a lower annealing temperature at second binding site 225c than a third, ligand-modified reverse primer. As used in this disclosure, the expression “annealing temperature” encompasses the maximum temperature at which a primer binds to the complementary region of the template DNA. Accordingly, when the temperature of the annealing stage of a PCR cycle utilizing both the second primer 210 and a ligand-modified primer is appropriately set, the ligand-modified third primer will bind more preferentially than the unmodified second primer. However, due to the much greater concentration of second primer 210 relative to the concentration of the third, ligand modified primer, additional copies of target DNA construct 235 (with regions 207a-207c and 225a-225c comprising completed adapters) will be produced. Thus, second primer 210 operates to ensure that a sufficient surplus of completed target DNA construct 235 is produced and that all of the ligand modified third primer is consumed during PCR. In this way, a normalized concentration of ligand modified amplicons may be obtained.

Referring to the example of FIG. 2D, a third concentration of a third primer 220 is added to the mixture containing the target DNA 201. According to various embodiments, the third primer 220 comprises a ligand modified reverse primer, which, unlike the second primer 210, contains a sequence that is complementary to second binding site 211 of target DNA 201. As shown in the example of FIG. 2D, the third primer 220 can comprise a second index sequence 225b (for example, an index sequence such as 115b in FIG. 1), and a second binding site 225c (for example, a binding site such as second capture sequence 110b in FIG. 1). Depending on embodiments, the third primer 220 has a length of between 18-65 base pairs, but other embodiments of greater or shorter length are possible and within the contemplated scope of this disclosure. As shown in the example of FIG. 2D, the third primer 220 further comprises a tagging ligand 230, which, according to various embodiments, is covalently bound to the third primer 220 and can bind to a magnetic affinity material (for example, streptavidin beads). In some embodiments, the tagging ligand 230 is biotin, which bonds strongly to streptavidin. However, embodiments using other ligands and modifiers used in attachment chemistry or affinity capture are within the contemplated scope of this disclosure. For example, instead of the tagging ligand 230, the third primer 220 may comprise an amino modified oligo that can survive PCR synthesis and that can later be attached to a solid surface, such as carboxylate modified magnetic beads. In this way, equal and determinate numbers of ligand-tagged constructs can be captured from each sample, based on the quantity/concentration of the third primer used.

As discussed elsewhere in this disclosure, to produce a normalized concentration of labeled constructs (for example, construct 100 in FIG. 1), the second primer 210 and the third primer 220 may be selected such that third primer 220 has a higher annealing temperature than the second primer 210, with the effect that third primer 220 binds preferentially relative to second primer 210. For example, in certain embodiments, the second primer has an annealing temperature between 60 and 64 degrees Celsius (° C.), while the third primer has an annealing temperature between 70° C. and 74° C. As such, the third primer 220 may preferentially bind to second binding site. Further, the relative concentrations of the second primer 210 and the third primer 220 are such that the second primer 210, while binding less preferentially than the third primer 220, is nonetheless sufficiently abundant to keep PCR amplification of the target DNA 201 on-going as to ensure that all of the third primer 220 is incorporated into copies of the target DNA 201.

Referring to the example of FIG. 2E, by performing PCR synthesis on an initial quantity of target DNA 201, a highly predictable quantity of ligand-tagged constructs 235 may be obtained. As shown in FIG. 2E, a ligand-tagged construct 235 comprises a sequencing-ready construct to which tagging ligand 230 is attached.

Given the strong affinity between streptavidin and biotin, all of the ligand-tagged constructs 235 can be captured from the rest of the sample by adding magnetic streptavidin affinity beads, applying a magnetic force to mechanically retain ligand-tagged constructs 235 within a container, and washing away remaining PCR reactants and products without an attached ligand. Ligand-tagged constructs 235 then can be released from the capture beads by performing a subsequent round of PCR synthesis. Alternative methods of capturing non-ligand modified constructs for sequencing include cleavage by restriction enzyme, denaturation by sodium hydroxide, or chemical reaction to disrupt attachment to the magnetic beads.

Table 1, below, illustrates an example of PCR cycling conditions for performing ligand-limited normalization (i.e., obtaining a determinate quantity of ligand tagged constructs proportional to an initial concentration of a ligand-modified primer), according to various embodiments of this disclosure. In the example, target DNA (for example, target DNA 201 in FIGS. 2A-2E or template DNA 105 in FIG. 1) can be obtained by performing an initial round of PCR (also referred to as “PCR1”) on a sample to obtain amplified quantities of target DNA. The product of PCR1 may then be combined with a first forward primer (for example, first primer 205 in FIG. 2B), a second, reverse primer (for example, second primer 210 in FIG. 2C), and a third, ligand-modified reverse primer (for example, third primer 220 in FIG. 2D). To ensure that the synthesis of ligand tagged constructs is ligand-limited, the concentrations of the first and second primers are selected to significantly exceed (for example, by being 10-50 times greater) that of the concentration of the third, ligand-modified primer. In certain embodiments, first and second primers are provided at normal PCR concentrations (for example, between 200 and 400 nM), while the ligand-modified primer is provided at a much lower concentration (for example, between 100 pM-10 nM). Furthermore, to ensure that the synthesis of ligand tagged constructs is ligand-limited, the second and third primers are designed such that the annealing temperature of the third primer is higher than that of the second primer. In some embodiments, the annealing temperature of the third primer may be 6-9° C. greater than that of the second primer. In certain embodiments, the annealing temperature of the third primer may be between 10-13° C. greater than that of the second primer. In various embodiments, the annealing temperature of the third primer may be 14° C., or more, greater than that of the second primer.

TABLE 1

| Step | Temp (° C.) | Time (Minutes) | Number of Cycles |
|---|---|---|---|
| Hot Start | 95 | 15:00 | 1 |
| Denaturation | 94 | 0:30 | 2 |
| Annealing | 57 | 0:30 | |
| Extension | 72 | 2:00 | |
| Annealing | 94 | 0:30 | 18 |
| Extension | 72 | 0:45 | |
| Final Extension | 72 | 2:00 | 1 |
| Hold (Storage) | 4 | | 1 |

As shown above, the relative concentrations of the first and second primers relative to the third primer ensures that the third primer is fully consumed within relatively few PCR cycles (for example, between 10-20 annealing-extension cycles). In certain embodiments, keeping the number of PCR cycles low may be advantageous in that it mitigates the formation of concatemers and unwanted “bubble products” (as used in this disclosure, “bubble products” encompasses mismatched amplicon complements), which can be produced when PCR reactants are substantially consumed.

As discussed herein with respect to FIGS. 2D and 2E, following PCR synthesis to generate a normalized quantity of ligand-tagged constructs, the constructs may be captured using magnetized streptavidin beads. A normalized quantity of sequencing-ready constructs from a plurality of individual samples may be obtained by performing a successive PCR step. Other alternative methods of retrieval of the normalized constructs from the magnetic capture beads are possible and contemplated elsewhere herein.

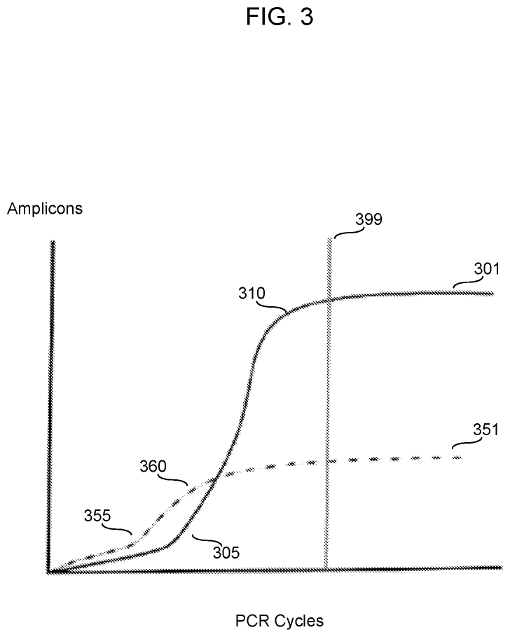

FIG. 3 illustrates a plot showing the relative amplification of ligand tagged amplicons (for example, ligand tagged construct 235 in FIG. 2E) relative to non-ligand tagged amplicons obtained when practicing ligand-limited normalizing PCR, according to certain embodiments of this disclosure. In the illustrative example of FIG. 3, two plots are shown, a first plot 301, shows a quantity of non-ligand tagged amplicons across PCR cycles (for example, the annealing-extension cycles described with reference to Table 1), wherein PCR is performed on mixture comprising an unspecified quantity of target DNA, a first, forward primer, a second, unlabeled (i.e., not ligand modified) reverse primer, and a third, ligand-modified reverse primer. FIG. 3 also shows a second plot 351, showing the quantity of ligand tagged amplicons during the same. Here, as in the example described with reference to Table 1, the relative concentrations of the first and second primers significantly exceed (for example, are between 10-50 times greater) than that of the third primer. Further, the third primer has a higher affinity for a binding site on the target DNA, and a higher annealing temperature than the second primer.

Referring to the example shown in FIG. 3, both the first plot 301 and the second plot 351 comprise a first inflection point (point 305 in first plot 301 and point 355 in second plot 351) where, after an initial warm-up period, exponential growth in the number of amplicons begins. As shown in FIG. 3, because the ligand-modified primer binds preferentially to a specified binding site (for example, second binding site 240b in FIG. 2E) of the target DNA compared to the unlabeled reverse primer, inflection point 355 occurs earlier than corresponding inflection point 305.

Further, as shown in the example of FIG. 3, both the first plot 301 and the second plot 351 include a second inflection point (point 310 in first plot 301 and point 360 in second plot 351), where exponential growth ceases and the growth in the number of amplicons diminishes across subsequent PCR cycles. As shown in FIG. 3, the second inflection point 360 for second plot 351 occurs earlier than corresponding second inflection point 310 in first plot 301. In practical terms, FIG. 3 illustrates that, at final PCR cycle 399 (for example, the 20^{th} PCR cycle), there have been sufficient PCR cycles between second inflection point 360 and final PCR cycle 399 to ensure that the ligand modified primer has been completely consumed, and that the number of ligand tagged constructs at the time of final PCR cycle 399 strongly corresponds to an initial number of strands of ligand modified primer (for example, third primer 220 in FIG. 2D).

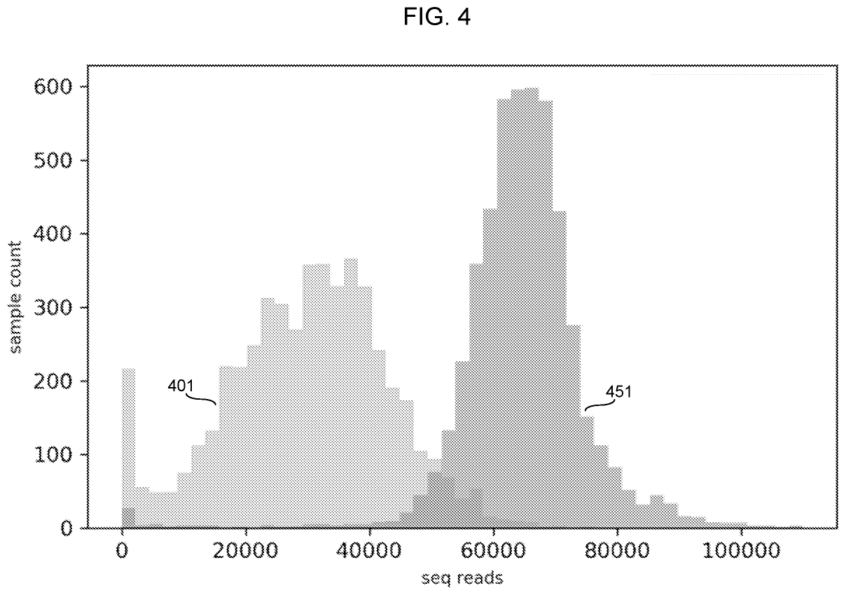

FIG. 4 illustrates a plot of two histograms showing the distribution of sequence reads of a first population of “raw” samples comprising a target DNA sequence (for example, target DNA 201 in FIG. 2A) following un-normalized PCR, and a second population of samples for which ligand-limited normalizing PCR according to various embodiments of this disclosure was performed prior to sequencing.

In the example shown in FIG. 4, the samples of the first population and the second population were obtained from initially equivalent (for example, a volume of sample divided into two smaller volumes, the first of which was added to the first sample population, and the second of which was added to the second sample population).

Referring to the example shown in FIG. 4, a first histogram 401 (shown in light grey) shows the distribution of samples relative to the number of sequences of target DNA that can be read in the sample. The first histogram 401 shows that, within the “raw” first sample population slightly more than 200 samples had between 0 and an initial threshold value of readable sequences of target DNA. Similarly, the first histogram 401 shows a broad distribution of sequence reads with a median value of approximately 35,000 reads per sample, with the upper tail of the distribution extending to approximately 60,000 reads per sample. Notably, the number of returned reads is not necessarily an indicator for normalization because simply loading more of the un-normalized population on the sequencer will result in more reads. The wide distribution among returned reads in histogram 401 illustrates the high degree of variability in non-normalized samples and the necessity of sample normalization when using multiple samples using next generation sequencing platforms.

By contrast, second histogram 451 shows that, in the second population, for which ligand-limited normalizing PCR, according to various embodiments of this disclosure, was performed, not only was the median sequence read higher—at around 70,000 sequence reads per sample, but the standard deviation of the distribution was much narrower than in the first population, with the majority of samples providing between 55,000-75,000 reads per sample. Further, in the second, normalized population, the number of samples with approximately zero reads was reduced approximately tenfold.

Accordingly, FIG. 4 illustrates the efficacy of embodiments according to this disclosure as a tool for obtaining normalized samples of sequencing-ready target DNA without the disadvantages of historical normalizing techniques.

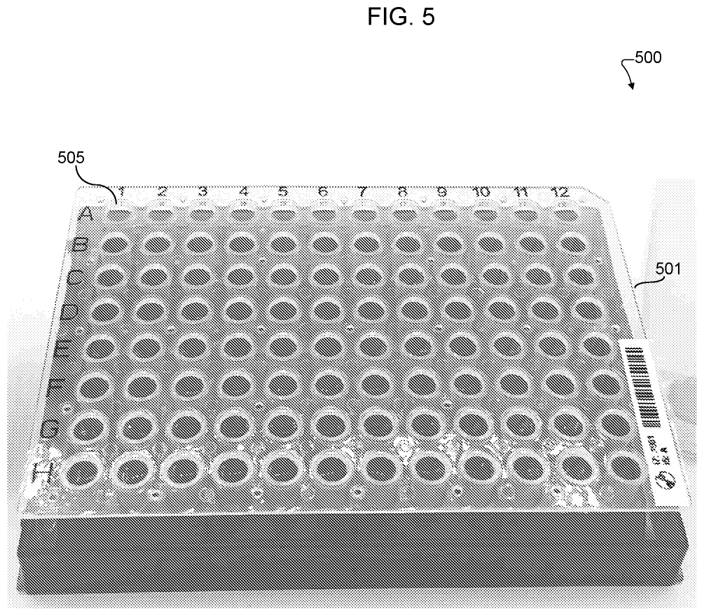

FIG. 5 illustrates an example of a normalization plate 500, according to various embodiments of this disclosure. The embodiment of the normalization plate 500 shown in FIG. 5 is for illustration only. Other embodiments could be used without departing from the scope of the present disclosure.

Referring to the explanatory example of FIG. 5, the normalization plate 500 includes a substantially planar member 501 comprising a plurality of sample wells (for example, sample well 505). In this example, normalization plate 500 includes 96 sample wells, though embodiments with greater or fewer sample wells are possible and within the contemplated scope of this disclosure.

Advantageously, in certain embodiments, certain of the reagents for performing ligand-limited normalization may be pre-measured and maintained stored in a dried sucrose solution in the sample wells of normalization plate 500 prior to normalization. In this way, normalization plate 500 can diminish the extent to which normalizing samples presents a bottleneck on sequencing throughput obtainable by next generation sequencers and other apparatus capable of parallel sequencing large numbers of samples.

According to various embodiments, each sample well of normalization plate 500 (including sample well 505) includes a volume of a dried sucrose solution, which depending on drying conditions, may be bound to the sample well. In the example of FIG. 5, the dried sucrose solution may include a first concentration of a forward primer (for example, first primer 205 in FIG. 2B), wherein the forward primer comprises a sequence of DNA (for example, a segment between 18-65 base pairs in length), which includes a region (for example, first binding site 207a in FIG. 2B) that is complementary to a first end of a template DNA segment. According to various embodiments, the dried sucrose solution further comprises a second primer, which is a ligand-modified reverse primer (for example, third primer 220 in FIG. 2D) that comprises a segment of DNA (for example, a segment between 18-65 base pairs in length) including a region (for example, second binding site 225a in FIG. 2D) that is complementary to a second end of the template DNA segment. According to various embodiments, the concentration of the forward primer within the dried sucrose solution is between 10-50 times greater than that of second primer. Further, the second primer is, in various embodiments, designed to have an annealing temperature that is higher than that of the first primer. For example, in certain embodiments, the first primer has an annealing temperature between 60° C. and 64° C., while the second primer has an annealing temperature between 70° C. and 74° C. Additionally, the dried sucrose solution includes a third primer, which is a non-ligand modified reverse primer that binds, albeit less preferentially, to the same binding site at the end of the template DNA as the second primer. In this example embodiment, the third primer, like the first primer, is provided at a concentration significantly exceeding that (for example, between 10-50×) of the second primer. As with the first primer, the third, added primer has an annealing temperature significantly lower (for example, between 6-14° C.) below that of the second primer.

To perform normalization, volumes of sample liquid containing template DNA are added to each sample well of normalization plate 500. An equal volume of 2×PCR master mix is then added to each well and thermal cycling is performed as shown in TABLE 1. Low reaction volumes are recommended (for example, 5 uL total PCR volume; 2.5 uL template DNA/2.5 uL 2×PCR mix) though larger and smaller reactions can be made to work as well.

With the template-DNA containing sample liquid added to each of the sample wells of normalization plate 500 along with a volume of PCR master mix, normalizing PCR (for example, as described with reference to FIGS. 2A-2E and Table 1) according to various embodiments of this disclosure may be performed.

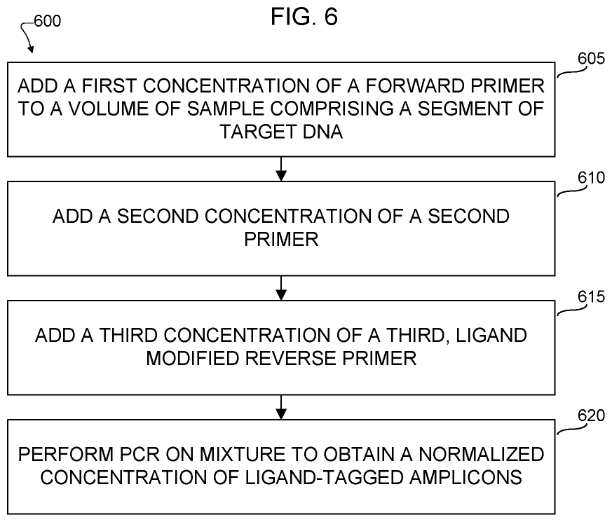

FIG. 6 illustrates operations of an example method 600 for performing ligand-limited normalizing PCR according to various embodiments of this disclosure. The operations described with reference to FIG. 6 may be performed at any suitable platform where the temperatures of the samples can be readily modulated to cycle the temperatures at which melting, annealing and extension occur. Examples of suitable platforms include, without limitation, normalization plates, such as the normalization plate 500 in FIG. 5.

Referring to the non-limiting example of FIG. 6, at operation 605, a first concentration of a forward primer (for example, first primer 205 in FIG. 2B, or a forward primer included in a dried sucrose mixture in a normalization plate) is combined with a volume of sample comprising segments of target DNA. According to various embodiments, the first concentration is in the range of 200-400 nM. The first primer comprises, at a minimum, a segment of DNA complementary to a first end of the target DNA. Depending on embodiments, the first primer can also include one or more of an index sequence (for example, first indexing sequence 115a in FIG. 1), and a capture sequence (for example, an Illumina P5 capture sequence).

As shown in the explanatory example of FIG. 6, at operation 610, a second concentration of a second primer (for example, second primer 210) is added to the mixture containing the target DNA and the first primer. In this example, the second concentration is selected to significantly exceed (for example, by a factor of 10-50×) that of the concentration of the third primer added at operation 615. According to various embodiments, the second primer is a reverse primer comprising a segment of DNA complementary to a second end (for example second binding site 225c in FIG. 2E) of the target DNA. Further, the second primer is selected to have a lower annealing temperature (for example, where the third primer has an annealing temperature between 70° C. and 74° C., the second primer may have an annealing temperature in the range of 60° C. to 64° C.).

At operation 615, a third concentration of a third, ligand-modified reverse primer (for example, third primer 220 in FIG. 2D) is added to the mixture. In some embodiments, the ligand-modified reverse primer comprises at a minimum, a segment of DNA complementary to a region of DNA at second end of the target DNA (for example, second binding site 211 in FIG. 2D) and a tagging ligand (for example, tagging ligand 230) to facilitate capture. In various embodiments, the ligand is biotin. In some embodiments, other ligands or complexes suitable for capture chemistry, such as an amino modified oligo which may survive PCR synthesis and which can later be attached to a solid surface, such as carboxylate modified magnetic beads, may be used.

Further, at operation 620, normalizing PCR cycle is performed, wherein the PCR cycle comprises an initial melt cycle, and successive annealing and extension cycles, wherein the annealing cycles are performed at a temperature closer to the annealing temperature of the third primer than that of the second primer. In this way, the third primer binds preferentially to the target DNA and third primer is fully consumed, as described with reference to the plots shown in FIG. 3 of this disclosure. Thus, the number of ligand-tagged constructs or amplicons resulting from the normalizing PCR is directly proportional to the quantity of ligand-modified third primer available for reaction. Provided that ligand-modified third primer can be provided in equal quantities across sample batches, the distribution of readable sequences per sample can be dramatically narrowed, as shown with reference to FIG. 4 of this disclosure.

None of the description in this application should be read as implying that any particular element, step, or function is an essential element that must be included in the claim scope. The scope of patented subject matter is defined only by the claims. Moreover, none of the claims is intended to invoke 35 U.S.C. § 112(f) unless the exact words “means for” are followed by a participle.

What is claimed is:

1. A method for obtaining a normalized quantity of DNA amplicons, the method comprising:
adding, to a volume of a sample comprising a target DNA segment, a first concentration of a forward primer, wherein the forward primer is complementary to a first end of the target DNA segment;
adding, to the volume of the sample, a second concentration of a second primer, wherein the second primer comprises an unlabeled reverse primer having a first annealing temperature, and a binding site specific to a complement of a third primer;
adding, to the volume of the sample, a third concentration of the third primer, wherein the third primer is a ligand modified reverse primer complementary to a second end of the target DNA segment and comprises a normalizing ligand,
wherein the third primer has a second annealing temperature, which is greater than the first annealing temperature,
wherein the third concentration is less than the second concentration; and
performing a polymerase chain reaction (PCR) on the volume of the sample to obtain a normalized concentration of ligand-tagged amplicons, wherein the PCR comprises one or more melt cycles, annealing cycles, and extension cycles,
wherein the annealing cycles are performed at a third annealing temperature, which is closer to the second annealing temperature than to the first annealing temperature.

2. The method of claim 1, wherein the normalizing ligand is biotin.

3. The method of claim 1, wherein the volume of the sample comprises a non-normalized concentration of multiplex PCR amplicons including amplicons of the target DNA segment,
wherein the ligand-tagged amplicons comprise constructs of DNA segments in the following order:
an Illumina P5 capture sequence;
a first index sequence;
an Illumina sequencing primer site;
the target DNA segment;
an Illumina paired-end sequencing site;
a second index sequence; and
an Illumina P7 capture sequence.

4. The method of claim 3, wherein the normalizing ligand is bound to the second primer.

5. The method of claim 1, wherein a ligand-tagged amplicon comprises:
a first adapter comprising an Illumina P5 binding site, an indexing sequence, and an Illumina Read1 primer site; and
a second adapter comprising an Illumina P7 binding site, an indexing sequence, and an Illumina Read2 primer site.

6. The method of claim 1, wherein the first annealing temperature is between 60 and 64 degrees Celsius.

7. The method of claim 1, wherein the second annealing temperature is between 70 and 74 degrees Celsius.

8. The method of claim 1, wherein a melt temperature is between 90 and 96 degrees Celsius.

9. The method of claim 1, wherein at least one of the first concentration and second concentrations are between 80-600 nanomolar (nM).

10. The method of claim 1, wherein the third concentration is between 8-12 nM.

11. The method of claim 1, further comprising:
subsequent to performing the PCR, adding magnetic affinity capture beads to the volume of the sample to bind with the ligand-tagged amplicons; and
applying a magnetic force, separating the ligand-tagged amplicons.

12. The method of claim 11, further comprising:
performing a second PCR (bead-release PCR) to capture an un-labeled population of the ligand-tagged amplicons from the magnetic affinity capture beads.

13. The method of claim 12, wherein the magnetic affinity capture beads are magnetic streptavidin beads.

14. A normalization plate comprising:
a substantially planar member comprising a plurality of sample wells, wherein each sample well of the plurality of sample wells comprises:
a volume of a dried sucrose solution bound to the sample well, the dried sucrose solution comprising:
a first concentration of a forward primer, wherein the forward primer is complementary to a first end of a target DNA segment; and
a second concentration of a second primer, wherein the second primer is a ligand modified reverse primer complementary to a second end of the target DNA segment, and wherein the second primer comprises a normalizing ligand,
wherein the second primer has a second annealing temperature, which is greater than an annealing temperature of a third primer.

15. The normalization plate of claim 14, wherein the normalizing ligand is biotin.

16. The normalization plate of claim 14, wherein the forward primer comprises an Illumina P5 binding site, an indexing sequence, and an Illumina Read1 primer site.

17. The normalization plate of claim 14, wherein the second primer comprises an Illumina P7 binding site, an indexing sequence, and an Illumina Read2 primer site.

18. The normalization plate of claim 14, wherein the normalizing ligand is bound to the second primer.

19. The normalization plate of claim 14, wherein the second annealing temperature is between 70 and 74 degrees Celsius.
